(19) United States
(12) Patent Application Publication
(10) Pub. No.: US 2025/0381228 A1
(43) Pub. Date: Dec. 18, 2025

(54) METHOD FOR TREATING OSTEOARTHRITIS WITH MESENCHYMAL STEM CELL EXOSOMES

(71) Applicant: Direct Biologics, LLC, Austin, TX (US)

(72) Inventors: Kenneth Allen Pettine, Fort Collins, CO (US); Timothy Alexander Moseley, Fallbrook, CA (US)

(21) Appl. No.: 19/256,922

(22) Filed: Jul. 1, 2025

Related U.S. Application Data
(62) Division of application No. 17/429,553, filed on Aug. 9, 2021.
(60) Provisional application No. 62/802,310, filed on Feb. 7, 2019.
(60) Provisional application No. 62/908,853, filed on Oct. 1, 2019.

(51) Int. Cl. A61K 35/26; A61K 9/00; A61K 35/28; A61P 19/02; A61P 29/00

(52) U.S. Cl. CPC A61K 35/26; A61K 9/0014; A61K 9/0019; A61K 35/28; A61P 19/02; A61P 29/00

(57) ABSTRACT

Disclosed herein are methods for treating osteoarthritis may be a one-step arthroscopic procedure and may include detaching synovial mesenchymal stem cells (MSCs) from the synovium using a brush device: covering articular cartilage in an affected joint with a scaffold, and placing concentrated MSC exosomes into the affected joint to stimulate differentiation of synovial MSCs into articular cartilage cells.

This application claims the benefit of U.S. Provisional Application No. 62/908,853, filed on October 1, 2019 and U.S. Provisional Application No. 62/802,310, filed on February 7, 2019, applications which are incorporated herein by reference in their entirety.

### I. BACKGROUND

Osteoarthritis (OA) is a disorder of any join in the body that leads to symptoms such as inflammation, pain, and limited function. It is also the most common chronic illness in the United States. Most arthritis occurs in the knee or hip joint, followed by the ankle and shoulder joint. In addition, arthritis of the fingers and base of the thumb is quite common.

OA is also called wear and tear arthritis, and it is by far the most common cause of arthritis. About a third of adults in the United States over 65 years have arthritis to the point of requiring regular medication. Symptoms include pain in a joint during or after movement, tenderness in the joint when pushed on, a crunching feeling or sound of bone rubbing on bone, and stiffness in the joint after periods of inactivity, such as sleeping or sitting.

The pathogenesis of knee OA have been linked to biomechanical and biochemical changes in the cartilage of the knee joint (e.g., the inability to withstand normal mechanical stresses, limited supply of nutrients and oxygen, inadequate synthesis of extracellular matrix components, increased synthesis of tissue-destructive proteinases, such as matrix metalloproteinases and aggrecanases, and overall apoptosis of chondrocytes). Recently, synovial inflammation has also been accredited as a factor limiting knee cartilage repair. Moreover, it correlates to clinical signs of knee OA, such as swelling of the knee and inflammatory pain. It is believed that synovial inflammation is a response of synovial macrophages to cartilage debris and catabolic mediators entering the synovial cavity.

Articular cartilage is both aneural and avascular. As such, cartilage is incapable of directly generating pain, inflammation, stiffness, or any of the symptoms that patients with OA typically describe. While the above is all known information, the exact causes of pain in OA are not entirely understood.

Therefore, what is needed is a method for restoring articular cartilage to a normal physiologic condition to stop synovial inflammation and, thus, eliminate the pain of OA.

### II. SUMMARY

Disclosed are methods and compositions related to mesenchymal stem cell (MSC) exosome compositions for use treating diseases, disorders, and injuries affecting joints.

In one aspect, disclosed herein are methods of treating, inhibiting, reducing, ameliorating and/or preventing a disease, disorder, injury (such as, for example, osteoarthritis, juvenile arthritis, psoriatic arthritis, infectious arthritis, rheumatoid arthritis, ankylosing spondylitis, gout, bursitis, tendinosis, tendonitis, sprain, labral tear, tear of a tendon, and/or tear of a ligament) or symptoms thereof (such as, for example, pain, inflammation, and/or swelling) affecting one or more joints (such as, for example, the ankle, knee, hip, writs, elbow, shoulder, knuckle, and/or neck) in a subject comprising administering to a subject a therapeutically effective amount of a mesenchymal stem cell (MSC) exosome preparation.

Also disclosed herein are methods of any preceding aspect, wherein the MSC exosome preparation is administered to each joint effected by the disorder, disease, or injury.

In one aspect, disclosed herein are methods of any preceding aspect, wherein the MSC exosome preparation further comprises growth factors (such as, for example, prostaglandin E2 (PGE2), transforming growth factor β1 (TGF-β1), hepatocyte growth factor (HGF), stromal cell derived factor-1 (SDF-1), nitric oxide, indoleamine 2,3-dioxygenase, interleukin-4 (IL-4), IL-6, interleukin-10 (IL-10), IL-1 receptor antagonist and soluble TNF-α receptor, insulin-like growth factors, fibroblast growth factors (FGF) 1-23 (especially, FGF1 and FGF2), bone morphogenetic proteins (BMPs) 1-15, epidermal growth factor (EGF), transforming growth factor-α (TGF-α) macrophage-stimulating protein (MSP), platelet derived growth factor (PLGF), vascular endothelial growth factor (VEGF), macrophage colony stimulating factor (M-CSF), insulin, granulocyte colony stimulating factor (G-CSF), granulocyte macrophage colony stimulating factor (GM-CSF), and/or hormones including estrogen, and thyroid hormones) obtained from MSC.

Also disclosed herein are methods of any preceding aspect, wherein the MSC exosomes are administered via injection, MSC exosome carrying scaffold, hydrogel, and/or topical cream or salve.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows a lower BPI and ODI=improvement, a higher LEFS score=improvement.

FIG. 2 shows Pre-procedure radiograph of shoulder showing Kellgren-Lawrence grade four osteoarthritis of the gleno-humeral joint.

FIG. 3 shows A lower BPI and QuickDash=improvement. A higher UFEI score=improvement.

FIG. 4 shows a diagram of the MSC exosome treatment for OA.

FIG. 5 shows a radiographs of a pelvis.

FIGS. 6A shows a radiograph of the Right Elbow shows Kellgren-Lawrence Grade Three changes of the Radio-ulnar joint and the humeral-ulnar joints.

FIG. 6B shows radiographs of the Left Elbow are Normal.

FIG. 7 shows range of motion in the elbow following EVIP administration.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10”as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to and 15 are considered disclosed as well as between 10 and 15. It is also understood that each unit between two particular units are also disclosed. For example, if 10 and 15 are disclosed, then 11, 12, 13, and 14 are also disclosed.

The term “subject” is defined herein to include animals such as mammals, including, but not limited to, primates (e.g., humans), cows, horses, pigs, sheep, goats, dogs, cats, rabbits, rats, mice and the like. In some embodiments, the subject is a human.

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intraarticular, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following me another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration but are undetectable or detectable at negligible amounts in distal parts of the subject's body. Administration includes self-administration and the administration by another.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, bot excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily, or the dose may be proportionally reduced as indicated by the exigencies of the therapeutic situation.

A “decrease” can refer to any change that results in a smaller gene expression, protein production, amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Treat,” “treating,” “treatment,” and grammatical variations thereof as used herein, include the administration of a composition with the intent or purpose of partially or completely preventing, delaying, curing, healing, alleviating, relieving, altering, remedying, ameliorating, improving, stabilizing, mitigating, and/or reducing the intensity or frequency of one or more a diseases or conditions, a symptom of a disease, disorder, injury, or condition, or an underlying cause of a disease or condition. Treatments according to the invention may be applied preventively, prophylactically, pallatively or remedially. Prophylactic treatments are administered to a subject prior to onset (e.g., before obvious signs of cancer), during early onset (e.g., upon initial signs and symptoms of cancer), or after an established development of cancer. Prophylactic administration can occur for day(s) to years prior to the manifestation of symptoms of an infection.

The terms “prevent,” “preventing.” “prevention,” and grammatical variations thereof as used herein, refer to a method of partially or completely delaying or precluding the onset or recurrence of a disease and/or one or more of its attendant symptoms or barring a subject from acquiring or reacquiring a disease or reducing a subject's risk of acquiring or reacquiring a disease or one or more of its attendant symptoms.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type 1 diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain (i.e., nociception) relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular MSC exosome (with or without growth factors) referred to herein as an extracellular vesicle isolate product (EVIP) is disclosed and discussed and a number of modifications that can be made to a number of molecules including the EVIP are discussed, specifically contemplated is each and every combination and permutation of EVIP and the modifications that are possible unless specifically indicated to the contrary. Thus, if a class of molecules A, B, and Care disclosed as well as a class of molecules D, E, and F and an example of a combination molecule, A-D is disclosed, then even if each is not individually recited each is individually and collectively contemplated meaning combinations, A-E, A-F, B-D, B-B, B-F, C-D, C-B, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

### B. Methods of Treating Osteoarthritis

In contrast to articular cartilage, the synovium and joint capsule are richly innervated and are likely the primary source of the pain in OA. The synovial reaction in OA includes synovial hyperplasia, fibrosis, thickening of the synovial capsule, activated synoviocytes and, in some cases, lymphocytic infiltrate (B- and T-cells, as well as plasma cells). The synovium is of obvious relevance as one of the most densely innervated structures of the joint. Synovial causes of pain include irritation of sensory nerve endings within the synovium from osteophytes and synovial inflammation that is due, at least in part, to the release of prostaglandins, leukotrienes, proteinases, neuropeptides, and cytokines. Pro-inflammatory examples include Interleukins 1, 6, and 8 along with various tumor necrosis factors. A semi-quantitative measure of synovitis from the infrapatellar fat pad is associated with pain severity. Any decrease in synovitis is associated with a decrease in OA pain severity.

Despite the fact that bone marrow is considered a well-accepted source of MSCs, various studies have reported that MSCs can be isolated from various adult mesenchymal tissues, including synovium. Studies have shown that synovium-derived MSCs have great proliferation potential and multilineage differentiation potential in vitro. Studies have also compared human MSCs derived from bone marrow, synovium, periosteum, adipose tissue, and muscle and determined that synovium-derived MSCs have greater expansion and chondrogenic ability in vitro than MSCS from other tissues. This suggests that synovium-derived MSCs are superior as a source for cartilage regeneration.

Studies have shown that synovial MSCs can be detached from the synovium using a brushing technique, resulting in a marked increase in the numbers of synovial MSCs found in the synovial fluid.

In one aspect, disclosed herein are methods of treating, inhibiting, reducing, ameliorating and/or preventing a disease, disorder, injury (such as, for example, osteoarthritis, juvenile arthritis, psoriatic arthritis, infectious arthritis, rheumatoid arthritis, ankylosing spondylitis, gout, bursitis, tendinosis, tendonitis, sprain, labral tear, tear of a tendon, and/or tear of a ligament) or symptoms thereof (such as, for example, pain, inflammation, and/or swelling) affecting one or more joints (such as, for example, the ankle, knee, hip, writs, elbow, shoulder, knuckle, and/or neck) in a subject comprising administering to a subject a therapeutically effective amount of a mesenchymal stem cell (MSC) exosome preparation.

In one aspect, disclosed herein are methods of treating, inhibiting, reducing, ameliorating and/or preventing a disease, disorder, injury (such as, for example, osteoarthritis, juvenile arthritis, psoriatic arthritis, infectious arthritis, rheumatoid arthritis, ankylosing spondylitis, gout, bursitis, tendinosis, tendonitis, sprain, labral tear, tear of a tendon, and/or tear of a ligament) or symptoms thereof (such as, for example, pain, inflammation, and/or swelling) affecting one or more joints (such as, for example, the ankle, knee, hip, writs, elbow, shoulder, knuckle, and/or neck) in a subject, wherein the MSC exosome preparation (also referred to herein as EVIP) further comprises growth factors (such as, for example, prostaglandin E2 (PGE2), transforming growth factor β1 (TGF-β1), hepatocyte growth factor (HGF), stromal cell derived factor-1 (SDF-1), nitric oxide, indoleamine 2,3-dioxygenase, interleukin-4 (IL-4), IL-6, interleukin-10 (IL-10), IL-1 receptor antagonist and soluble TNF-α receptor, insulin-like growth factors, fibroblast growth factors (FGF) 1-23 (especially, FGF1 and FGF2), bone morphogenetic proteins (BMPs) 1-15, epidermal growth factor (EGF), transforming growth factor-α (TGF-α) macrophage-stimulating protein (MSP), platelet derived growth factor (PLGF), vascular endothelial growth factor (VEGF), macrophage colony stimulating factor (M-CSF), insulin, granulocyte colony stimulating factor (G-CSF), granulocyte macrophage colony stimulating factor (GM-CSF), and/or hormones including estrogen, and thyroid hormones) obtained from MSC.

It is understood and herein contemplated that the disclosed MSC exosome treatments may not be curative of a disease, disorder, injury, or condition to a joint and may reduce or inhibit the injury, disease, or disorder. In one aspect, the MSC exosome preparation decreases symptoms of a disease, disorder, and/or injury (such as, for example, pain, inflammation, and/or swelling) rather than being curative or repairing the disease, disorder, or injury. Thus, in one aspect, disclosed herein are methods of treating, inhibiting, reducing, preventing and/or ameliorating pain, inflammation, and/or swelling associated with a disease, disorder, and/or injury affecting one or more joints of a subject comprising administering to the subject any of the MSC exosome preparations disclosed herein (in some cases including MSC derived growth factors).

It is understood and herein contemplated that administration can be directly to one or more effected joints. As noted throughout, administration of the disclosed MSC derived exosomes and/or growth factors can be any method know to those of skill in the art. Accordingly, disclosed herein are methods of treating, inhibiting, reducing, ameliorating and/or preventing a disease, disorder, injury (such as, for example, osteoarthritis, juvenile arthritis, psoriatic arthritis, infectious arthritis, rheumatoid arthritis, ankylosing spondylitis, gout, bursitis, tendinosis, tendonitis, sprain, labral tear, tear of a tendon, and/or tear of a ligament) or symptoms thereof (such as, for example, pain, inflammation, and/or swelling) affecting one or more joints (such as, for example, the ankle, knee, hip, writs, elbow, shoulder, knuckle, and/or neck) in a subject comprising administering to a subject a therapeutically effective amount of a mesenchymal stem cell (MSC) exosome preparation, wherein the MSC exosomes are administered via injection. MSC exosome carrying scaffold, hydrogel, and/or topical cream or salve. As the field of tissue engineering progresses, the need for novel scaffold structures and reproducible fabrication techniques has become of paramount importance. The use of biodegradable polymers, such as poly lactic acid (PLA), has become widespread, but the manner in which these polymers are processed, and the additives used at the time of manufacture, allows the final properties of the scaffold to be tailored.

Poly-hydroxyl acids, such as PLA and poly lactic-co-glycolic acid (PLGA), have been extensively used for tissue engineering procedures, as these materials bulk-degrade by hydrolysis, providing a controllable drug release and degradation profile to match tissue in-growth. With careful use of molecular weights, cross links and side chains, materials can be produced with tailor-made properties making them ideal for use in tissue engineering matrices. Furthermore, poly-hydroxyl acid materials also have a long history of in vivo usage as degradable sutures, drug delivery devices and biodegradable surgical components.

Existing scaffold types include high-pressure, CO₂ foamed scaffolds, injectable scaffolds, and novel custom scaffolds. These can be further modified using growth factors, zonation of materials, and plasma polymerization deposition. While the scaffold enhances residence of the synovial MSCs into being adjacent to the articular cartilage, this can be augmented by the addition of cytokines. For instance, PLGA with transforming growth factor-Bβ3 (TFG-β3) enhances MSC differentiation into chondrocytes, while implantation of PLGA with stromal-derived factor-1α (SDF-1α) results in repair of the articular cartilage. Thus, implantation of PIGA combined with various cytokines enhances more efficient differentiation of synovial MSCs into articular cartilage. This technique provides concentrated MSC growth factors and RNA to the synovial MSCs located in the peri-articular scaffold to maximize differentiation into chondrocytes.

By way of example, some embodiments of the invention include a method for treating osteoarthritis, the method comprising a one-step arthroscopic procedure to restore arthritic articular cartilage to a normal physiologic condition. Specifically, the method may comprise detaching synovial mesenchymal stem cells (MSCs) from the synovium using a brush device; covering the articular cartilage with a scaffold; and placing concentrated MSC exosomes into the affected joint, such as the knee joint, to stimulate the differentiation of the synovial MSCs into articular cartilage cells.

In embodiments, the brush may be an arthroscopic brush specifically designed to be used in conjunction with a particular joint.

When the scaffold is placed arthroscopically into the joint, such as the knee, to cover the articular cartilage, the intraarticular floating MSCs may attach to the peri-articular scaffold. Placing the MSC exosomes into the joint may provide growth factors and various RNAs to stimulate the differentiation of the synovial MSCs into chondrocytes. As a result, the damaged arthritic articular cartilage may be restored.

### C. Mesenchymal Stem Cells

As noted throughout, the treatment compositions disclosed herein can utilize exosomes and/or growth factors derived from mesenchymal stem cells (MSCs). While existing autogenous and allogeneic MSCs contained within bone marrow, bone marrow concentrate, synovia-derived mesenchymal stem cells (MSCs), or adipose-derived stromal vascular fraction (SVF) or various post-natal products from umbilical cord, placenta or amnion, expanded MSC cultures are currently being used to treat wounds, orthopedic pathology, and spine pathology; the existing treatments do not contain large amounts of MSC secretomes (including, but not limited to growth factors, cytokines, chemokines, exosomes, extracellular vesicles, and/or extracts). Additionally, despite evidence in the art that treatments comprising stem cells (including injectable treatments) can help prevent aging and treat scarring, uneven pigmentation, existing skin products, such as creams, lotions, serums, make-up, and the like, while including ingredients that potentially help treat and strengthen the skin, other topical products do not penetrate the epidermis and more importantly do not include human MSCs, or MSC-derived growth factors and proteins. In fact, prior to the present disclosure an active MSC growth factor product that can be used for these applications has not been developed. Thus, in one aspect, disclosed herein are MSC secretome compositions (including, but not limited to MSC growth factor, MSC exosome, MSC extracts and/or extracellular vesicle comprising compositions) for use in the treatment of wounds, orthopedic disorders, orthopedic injuries, ophthalmology, spinal injury, or spinal disorders, said treatment compositions comprising (i) a growth factor powdered additive comprising a mesenchymal stem cell (MSC) derived preparation and (ii) a pharmaceutically acceptable carrier.

As noted above, MSC are multipotent cells that have the ability to differentiate into a multitude of cell types including myocytes, chondrocytes, adipocytes, and osteoblasts, Typically, these cells can be found in the placenta, umbilical cord blood, adipose tissue, bone marrow, or amniotic fluid, including perivascular tissue. As used herein, “MSC” refers to non-terminally differentiated cells including but not limited to multipotential stem cell, multipotential stromal cell, stromal vascular cells, pericytes, perivascular cells, stromal cells, pluripotent cells, multipotent cells, adipose-derived fibroblast-like cells, adipose-derived stromal vascular fraction, adipose-derived MSC, bone marrow-derived fibroblast-like cells, bone marrow-derived stromal vascular fraction, bone marrow-derived MSC, tissue-derived fibroblast-like cells, adult stem cells, adult stromal cells, keratinocytes, and/or melanocytes.

It has been long recognized that MSC, in addition to their differentiation potential, have the immunomodulatory abilities resulting in the expression of many different cytokines and growth factors. As used herein, a “MSC preparation” or “MSC secretome composition” refers to a composition comprising MSC growth factors. MSC exosomes, extracellular vesicles, or acellular extracts of MSCs or MSC lysates obtained from human MSCs, fibroblast-like cells, and non-human animal MSCs including, but not limited to MSCs from horses, cows, pigs, sheep, non-human primates, dogs, cats, rabbits, rats, and mice. In embodiments, the MSCs may be derived from the patient to which the composition will be applied (autologous) or derived from another individual (allogeneic). The MSCs may be culture expanded to collect the conditioned media or to increase the quantity of cells for the lysate or used freshly prior to incorporation into the composition of the present disclosure.

The MSC secretome compositions (including, but not limited to MSC growth factor, MSC exosome, MSC extracts and/or extracellular vesicle comprising compositions) may comprise about 0.00001 to about 20 wt. %, such as from about 0.01 to about 10 wt. %, of a mesenchymal stem cell (MSC) extract. MSC exosome, or MSC growth factor preparation. The MSC preparation may comprise either MSC conditioned media or MSC lysate from cell culture expanded MSCs. In some embodiments, the composition may further comprise from about 0.01 to about 10 wt. % of a cell-free medium conditioned by growth of MSCs or MSC lineage cells, wherein the cells are cultured under normal hyperoxyic culturing conditions or under artificial wound healing conditions.

As disclosed herein the MSCs used to produce the disclosed MSC additives (including growth factor secretome composition either frozen or powdered additives) can be selectively stimulated to produce MSC growth factors, secretomes, cytokines, chemokines, mesenchymal stem cell proteins, peptides, glycosaminoglycans, extracellular matrix (ECM), proteoglycans, secretomes, and exosomes. As used herein, MSC growth factors include but are not limited to prostaglandin E2 (PGE2), transforming growth factor β1 (TGF-β1), hepatocyte growth factor (HGF), stromal cell derived factor-1 (SDF-1), nitric oxide, indoleamine 2,3-dioxygenase, interleukin-4 (IL-4), IL-6, interleukin-10 (IL-10), IL-1 receptor antagonist and soluble TNF-α receptor, insulin-like growth factors, fibroblast growth factors (FGF) 1-23 (especially, FGF1 and FGF2), bone morphogenetic proteins (BMPs) 1-15, epidermal growth factor (EGF), transforming growth factor-α (TGF-α) macrophage-stimulating protein (MSP), platelet derived growth factor (PLGF), vascular endothelial growth factor (VEGF), macrophage colony stimulating factor (M-CSF), insulin, granulocyte colony stimulating factor (G-CSF), granulocyte macrophage colony stimulating factor (GM-CSF), as well as hormones including estrogen, and thyroid hormones.

In one aspect, the MSC preparation (such as, for example, a MSC secretome composition) comprises MSC growth factors, MSC exosomes, and/or cellular extracts of MSCs or MSC lysates obtained from MSCs cultured under standard hyperoxyic culturing conditions (for example, 21% oxygen) or MSCs cultured under artificial wound healing conditions (such as, for example, 0.1% to about 5% oxygen in the presence of inflammatory cytokines, angiogenic factors, and reduced glucose).

As disclosed herein artificial wound healing conditions simulate growth conditions in real wounds where there is a reduction in nutrient supply and redaction of waste removal that is usually caused by a disruption in local blood circulation. This creates a harsh environment for cells until new blood vessels are created and blood circulation is restored. Accordingly, artificial wound healing conditions used to culture MSCs can include one or more of the following growth conditions reduction in glucose availability, reduction in oxygen tension, reduction in pH, and increased temperature.

In one aspect, the glucose availability can be reduced relative to normal control. Modified culture media to reduce glucose, but not damage the cells can be between 0 and 50% reduction in glucose, more preferably between about 5% and 40% reduction in glucose. For example. MSC artificial wound healing culture conditions can comprise glucose reduction of about 1, 2, 3, 4, 5, 6,7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50% such as a glucose reduction from about 5% to about 15%, from about 10% to about 20%, from about 15% to about 25%, from about 20% to about 30%, or from about 25% to about 35%.

In one aspect, oxygen tension can be reduced to oxygen levels to hypoxic conditions. Normal atmospheric oxygen is approximately 21% and any reduction is considered hypoxic. Thus, in one aspect, MSCs can be cultured at between 0.0% and 20.9% oxygen, from about 0.1% to about 0.5% oxygen, from about 0.1% to about 2.0%, from about 0.1% to about 5.0% oxygen, from about 0.5% to 5.0%, from about 1.0% to about 10% oxygen, about 5.0% to about 10.0% oxygen; and from about 10.0% to about 15.0% under artificial wound healing conditions. Preferably during MSC would healing culture conditions oxygen tension is between about 0.5% and 20.5% oxygen, such as, for example, 0, 0.05, 0.1, 0.2, 0.3, 0.4, 0.5, 0.6, 0.7, 0.8, 0.9, 1.0, 1.1, 1.2, 1.3, 1.4, 1.5, 1.6, 1.7, 1.7, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4,6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 10.5, 11, 11.5, 12, 12.5, 13, 13.5, 14, 14.5, 15, 15.5, 16, 16.5, 17, 17.5, 18, 18.5, 19, 19.5, 20, or 20.5% oxygen.

The pH can also be reduced under artificial wound healing conditions. Physiologic pH is maintained very tightly and is usually very close to a neutral pH=7.2±0.2 (7.0−7.4). However, in a wound the acidic environment can have a pH=6.2±0.2 (i.e., a pH from 6.0 to about 6.4). Thus, under artificial wound healing culture conditions, pH can be from about 6.0 to about 7.4, for example, from 6.0 to about 6.4, from about 6.2 to about 6.4, from about 6.2 to about 6.6, from about 6.4 to about 6.6, from about 6.4 to about 6.8. or from about 6.6 to about 7.0, such as 6.0, 6.1, 6.2, 6.3, 6.4, 6,5, 6.6, 6.7, 6.8, 6.9, 7.0, 7.1, 7.2, 7.3 or 7.4.

Under artificial wound healing culture conditions, the temperature of the culture environment may be raised to simulate temperature increases at the site of a wound. Physiologic homeostasis temperature is maintained at 37° C. (98.6° F.). A slight increase or decrease can cause significant changes to cellular metabolism. By increasing the temperature above 37° C. to any temperature up to about 40° C. (104° F.) can create an “feverous” environment. Thus, in on aspect, the artificial wound healing culture conditions for the MSCs can comprise from about 35° C. to about 39° C. , from about 35° C. to about 36° C. , from about 36° C. to about 37° C., from about 37° C. to about 38° C., from about 38° C. to about 39° C., from about 39° C. to about 40° C. In one aspect, the temperature of the artificial wound healing culture can be 35.0, 35.1, 35.2, 35.3, 36.4, 35.5, 35.6, 35.7, 35.8, 35.9, 36.0, 36.1, 36.2, 36.3, 36.4, 36.5, 36.6, 36.7, 36.8, 36.9, 37.0, 37.1, 37.2, 37.3, 37.4, 37.5, 37.6, 37.7, 37.8, 37.9, 38.0, 38.1, 38.2, 38.3, 38.4, 38.5, 38.6, 38.7, 38.8, 38.9, 39.0, 39.1, 39.2, 39.3, 39.4, 39.5, 39.6, 39.7, 39.8, 39.9, or 40.0° C.

In one aspect, the MSC secretome compositions (including, but not limited to MSC growth factor, MSC exosome, MSC extracts and/or extracellular vesicle comprising compositions) can further comprise a protective coating (such as, for example, a cryoprotectant oligosaccharide and a protein solution) to reduce degradation of the growth factors. It is understood and herein contemplated that the protective coating can be engineered as a polymer. “Polymer” refers to a relatively high molecular weight organic compound, natural or synthetic, whose structure can be represented by a repeated small unit, the monomer. Non-limiting examples of polymers include polyethylene, rubber, cellulose. Synthetic polymers are typically formed by addition or condensation polymerization of monomers. The term “copolymer” refers to a polymer formed from two or more different repeating units (monomer residues). By way of example and without limitation, a copolymer can be an alternating copolymer, a random copolymer, a block copolymer, or a graft copolymer. It is also contemplated that, in certain aspects, various block segments of a block copolymer can themselves comprise copolymers. The term “polymer” encompasses all forms of polymers including, but not limited to, natural polymers, synthetic polymers, homopolymers, heteropolymers or copolymers, addition polymers, etc. In one aspect, the gel matrix can comprise copolymers, block copolymers, diblock copolymers, and/or triblock copolymers.

In one aspect, the protective coating can comprise a biocompatible polymer. In one aspect, biocompatible polymer can be crosslinked. Such polymers can also serve to slowly release the adipose browning agent and/or fat modulating agent into tissue. As used herein biocompatible polymers include, but are not limited to polysaccharides; hydrophilic polypeptides; poly(amino acids) such as poly-L-glutamic acid (POS), gamma-polyglutamic acid, poly-L-aspartic acid, poly-L-serine, or poly-L-lysine; polyalkylene glycols and polyalkylene oxides such as polyethylene glycol (PEG), polypropylene glycol (PPG), and poly(ethylene oxide) (PEO); poly(oxyethylated polyol); poly(olefinic alcohol); polyvinylpyrrolidone); poly(hydroxyalkylmethacrylamide); poly(hydroxyalkylmethacrylate); poly(saccharides); poly(hydroxy acids); poly(vinyl alcohol), polyhydroxyacids such as poly(lactic acid), poly(gly colic acid), and poly(lactic acid-co-glycolic acids); polyhydroxyalkanoates such as poly3-hydroxybutyrate or poly4-hydroxybutyrate; polycaprolactones; poly(orthoesters); polyanhydrides; poly(phosphazenes); poly(lactide-co-caprolactones); polycarbonates such as tyrosine polycarbonates; polyamides (including synthetic and natural polyamides), polypeptides, and poly(amino acids); polyesteramides; polyesters; poly(dioxanones); poly(alkylene alkylates); hydrophobic polyethers; polyurethanes; polyetheresters; polyacetals; polycyanoacry lates; polyacrylates; polymethylmethacrylates; polysiloxanes; poly(oxyethylene)/poly(oxypropylene) copolymers; polyketals; polyphosphates; polyhydroxyvalerates; polyalkylene oxalates; polyalkylene succinates; poly(maleic acids), as well as copolymers thereof. Biocompatible polymers can also include polyamides, polycarbonates, polyalkylenes, polyalkylene glycols, polyalkylene oxides, polyalkylene terepthalates, polyvinyl alcohols (PVA), methacrylate PVA (m-PVA), polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethylmethacry late), poly(butylmethacrylate), poly(isobutylmethacry late), poly(hexlmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), poly(vinyl acetate, poly vinyl chloride polystyrene and polyvinylpryrrolidone, derivatives thereof, linear and branched copolymers and block copolymers thereof, and blends thereof. Exemplary biodegradable polymers include polyesters, poly(ortho esters), poly(ethylene amines), poly(caprolactones), poly(hydroxybutyrates), poly(hydroxyvalerates), polyanhydrides, poly(acrylic acids), polyglycolides, poly(urethanes), polycarbonates, polyphosphate esters, polyphospliazenes, derivatives thereof, linear and branched copolymers and block copolymers thereof, and blends thereof.

In some embodiments the protective coating comprises carbohydrate construction of monosaccharides as well as carbohydrate polymers such as disaccharides or polysaccharides including but not limited to non reducing poly or disaccharides as well as any combination thereof. Examples of carbohydrates that can be used in the protective coating comprise Glucose, Aldoses (D-Allose, D-Altrose, D-Mannose, etc.), Glucopyranose, Pentahydroxyhexanal, α-D-Glucopyranosyl-D-glucose, α-D-Glacopyranosyl-dihydrate, Polymer of β-D-Glycopyranosyl units, β-D-Fructofuranosyl α-D-glucopyranoside (anhydrous/dihydrate), β-D-Galactopyranosyl-D-glucose, α-D-Glucopyranosyl-α-D-glucopyranoside (anhydrous/dihydrate), Galactose, Pentoses (Ribose, xylose, lyxose), Dextrose, Dodecacarbon monodecahydrate, Fructose, Sucrose, Lactose, Maltose, Trehalose, Agarose, D-galactosyl-β-(1-4)-anhydro-L-galactosyl, Cellulose, Polymer of β-D-Glycopyranosyl units, and Starch, as well as, Polyhydric alcohols, Polyalcohols, Alditols, Erythritol, Glycitols, Glycerol, Xylitol, and Sorbitol.

In some embodiments the protective coating contains biocompatible and/or biodegradable polyesters or polyanhydrides such as poly(lactic acid), poly(glycolic acid), and poly(lactic-co-glycolic acid). The particles can contain one more of the following polyesters: homopolymers including glycolic acid units, referred to herein as “PGA”, and lactic acid units, such as poly-L-lactic acid, poly-D-lactic acid, poly-D,L-lactic acid, poly-L-lactide, poly-D-lactide, and poly-D-L-lactide5 collectively referred to herein as “PLA”, and caprolactone units, such as poly(e-caprolactone), collectively referred to herein as “PCL”; and copolymers including lactic acid and glycolic acid units, such as various forms of poly(lactic acid-co-glycolic acid) and poly(lactide-co-glycolide) characterized by the ratio of lactic acid:glycolic acid, collectively referred to herein as “PLGA”; and polyacrylates, and derivatives thereof. Exemplary polymers also include copolymers of polyethylene glycol (PEG) and the aforementioned polyesters, such as various forms of PLGA-PEG or PLA-PEG copolymers, collectively referred to herein as “PEGylated polymers”. In certain embodiments, the PEG region can be covalently associated with polymer to yield “PEGylated polymers” by a cleavable linker. In one aspect, the polymer comprises at least 60, 65, 70, 75, 80, 85. 89, 90. 91. 92. 93, 94, 95, 96, 97, 98, or 99 percent acetal pendant groups.

The triblock copolymers disclosed herein comprise a core polymer such as, example, polyethylene glycol (PEG), polyvinyl acetate, polyvinyl alcohol, polyvinyl pyrrolidone (PVP), polyethyleneoxide (PEO), poly vinyl pyrrolidone-co-vinyl acetate), polymethacrylates, polyoxyethylene alkyl ethers, polyoxyethylene castor oils, polycaprolactam, polylactic acid, polyglycolic acid, poly(lactic-glycolic) acid, poly(lactic co-glycolic) acid (PLGA), cellulose derivatives, such as hydroxymethylcellulose, hydroxypropylcellulose and the like.

Examples of diblock copolymers that can be used in the protective coatings disclosed herein comprise a polymer such as, example, polyethylene glycol (PEG), polyvinyl acetate, polyvinyl alcohol (PVA), polyvinyl pyrrolidone (PVP, polyethyleneoxide (PEO), poly(vinyl pyrrolidone-co-vinyl acetate), polymethacrylates, polyoxyethylene alkyl ethers, polyoxyethylene castor oils, polycaprolactam, polylactic acid, polyglycolic acid, poly(lactic-glycolic; acid, poly(lactic co-glycolic) acid (PLGA).

In one aspect, the protective coating contains (i.e., the encapsulated, the encapsulated compositions can further comprise lecithin or hydrolyzed lecithin as a carrier or as encapsulation material. As used herein, lecithin and/or hydrolyzed lecithin coatings include coatings comprising phosphatidyl choline, phosphatidyl inositol, phosphatidyl ethanolamine, phosphatidylserine, and phosphatidic acid. Sources of the lecithin can be plat or animal sources.

In one aspect, any of the polymers, monosaccharides, disaccharides, of polysaccharides used to form the protective coating formed by placing the MSC additive in a encapsulating solution can be at an appropriate concentration for form the protective coating. For example, polymers, monosaccharides, disaccharides, or polysaccharides can be at any concentration between 0.01 mM and 10.0 M concentration, for example, from about. 0.01 M to about 0.1 M, from about 0.1 mM to about 1.0 M, from about 1.0 M to about, 10.0 M. Exemplary concentrations include 0.01, 0.02, 0.03, 0.04, 0.05, 0.06, 0.07, 0.08, 0.09, 0.1, 0.2, 0.3, 0.4, 0.4, 0.6, 0.7, 0.8, 0.9, 1, 2, 3, 4, 5, 6, 7, 8, 9, 10, 20, 30, 40, 50, 60, 70, 80, 90, 100, 110, 120, 130, 140, 150, 160, 170, 180, 190, 200, 225, 250, 275, 300, 325, 350, 375, 400, 450, 500, 600, 700, 800, 900 mM, 1, 1.1, 1.2, 1.3, 1.4, 1.5, 1.6, 1.7, 1.8, 1.9, 2, 3, 4, 5, 6, 7, 8, 9, 10 M.

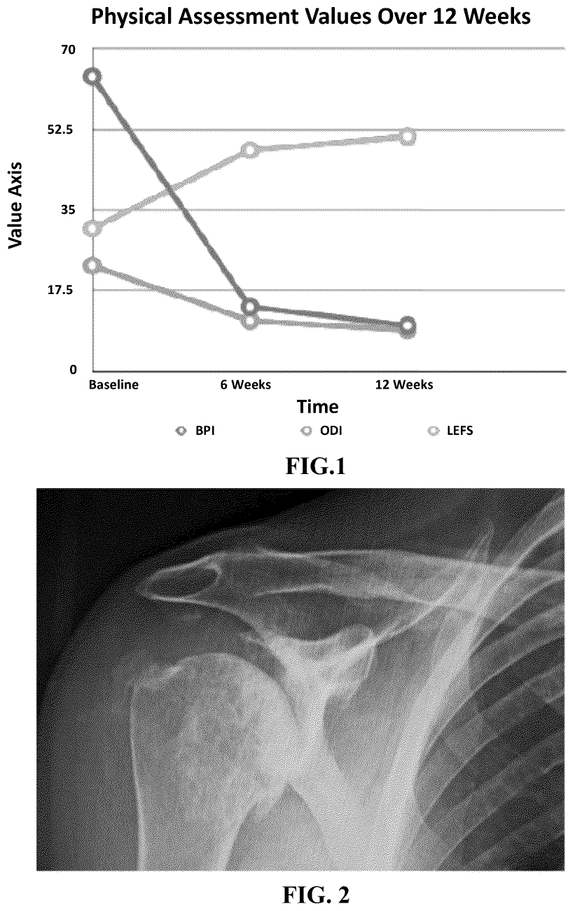

As shown in FIGS. 1 and 2, the exosomses and extracellular vesicles in the disclosed MSC secretome compositions have been produced.

In one aspect, it is understood and herein contemplated that one way to treat a wound is through administration of the MSC secretome compositions (including, but not limited to MSC growth factor, MSC exosome, MSC extracts and/or extracellular vesicle comprising compositions) subcutaneously, intramuscularly, intravenously, topically (such as, for example, through the use of salves, creams, and/or ointments), but also by impregnating stents, sponges, matrixes, scaffolds, bandages, dressing, sutures, grafts, surgical drapes, surgical adhesive, and/or staples with the MSC secretome compositions. Thus, in one aspect, disclosed herein are medicated stents, scaffolds, sponges, matrixes, adhesive bandages, wound dressings, grafts, surgical drapes, sutures, salves, creams, or wound adhesives comprising a therapeutically effective amount of the MSC secretome composition. The MSC secretome compositions (including, but not limited to MSC growth factor, MSC exosome, MSC extracts and/or extracellular vesicle comprising compositions), as noted above, can be administered topically and applied to the face, the neck, the hands, or any other desired part of the body. When applied to an adhesive bandage, wound dressing, grafts, surgical drape, suture, scaffold, matrix, sponge, or stent, the MSC secretome composition can be a applied as a powder.

In one aspect, the MSC secretome compositions (including, but not limited to MSC growth factor, MSC exosome, MSC extracts and/or extracellular vesicle comprising compositions) disclosed herein may comprise any known ingredients typically found in the wound healing fields, such as oils, waxes or other standard fatty substances, or conventional gelling agents and/or thickeners; emulsifiers; moisturizing agents; emollients; sunscreens; hydrophilic or lipophilic active agents, such as ceramides: agents for combating free radicals; bactericides; sequestering agents; preservatives; basifying or acidifying agents; fragrances; surfactants; fillers; natural products or extracts of natural product, such as aloe or green tea extract; vitamins; or coloring materials. Other ingredients that may be combined with the powder may include an antioxidant, which can be selected from a variety of antioxidants. Suitable antioxidants include vitamins, such as Vitamin C (L-Ascorbate, Ascorbate-2 Phosphate magnesium salt, Ascorbyl Palmitate, Tetrahexyldecyl Ascorbate), Vitamin E (Tocotrienol), Vitamin A (retinol, retinal, retinoic acid, provitamin A carotenoids, such as beta-carotene), N-acetyl glucosamine, or other derivatives of glucosamine. Other ingredients may include at least one essential fatty acid, such as (Ω-3, Ω-6, and Ω-9 polyunsaturated fatty acids, such as linoleic acid (LA), gamma-linoleic acid (GLA), alpha-linoleic acid (ALA), dihomo-y-linolenic acid (DGLA), arachidonic acid (ARA), and others. The fatty acids may be derived from various sources including evening primrose oil, black currant oil, borage oil, or GLA modified safflower seeds. Other ingredients may include a platelet rich fibrin matrix, at least one ingredient to support ECM production and production of hyaluronic acid, such as N-acetyl glucosamine or other derivatives of glucosamine, ultra-low molecular weight (ULMW) hyaluronic acid, chondroitin sulfate, or keratin sulfate.

It is understood and herein contemplated that the MSC secretome compositions disclosed herein can provide wound healing rejuvenation, augmentation, and improved or restored skin tissue. The composition may also be used as an injectable in the treatment of joint arthritis and degenerated spinal discs. Moreover, embodiments of the composition may not require the inclusion of additional growth factors or hormones, such as insulin, insulin-like growth factors, thyroid hormones, fibroblast growth factors, estrogen, retinoic acid, and the like. In some aspect, the disclosed stem cell growth factor compositions can comprise additional active ingredients including, but not limited to antibiotics, anti-acne agents, liposomes, antioxidants, platelet-rich fibrin matrixes, analgesic, anti-inflammatories well as, additional growth factors, such as insulin, insulin-like growth factors, thyroid hormones, fibroblast growth factors, estrogen, retinoic acid, and the like. Such additional active ingredients can be mixed with the stem cell growth factor and extracellular vesicle compositions disclosed herein as well as MSC conditioned media, MSC lystates, and MSC-derived produces and then thawed or dissolved, mixed, or suspended in a mixture of emulsifying lanolin alcohols, waxes, and oils or a mixture of petrolatum or mineral oil, a quaternary ammonium compound, a fatty alcohol, and a fatty ester emollient, or lotions that are substantially similar in composition.

### D. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1: Intra-Articular Injection of an Extracellular Vesicle Isolate Product to Treat Hip Labral Tears

Diarthrodial joints, such as the knee, hip, and shoulder consist of articular cartilage, a synovial capsule, and a fibrocartilaginous structure to increase the stability of the joint. The knee has both a medial and lateral meniscus. The patient's shoulder and hip each have a labrum which increases the concavity of the glenoid and acetabulum. These fibrocartilaginous structures all have a nerve supply and when torn, can be very painful. These structures have a limited blood supply and when damaged, have minimal capacity to heal. These structures can tear with an acute injury or by a chronic degenerative process. The acetabular labrum creates a fibrocartilaginous seal around the osseous acetabulum. The labrum increases the depth, surface area, volume, congruity, and stability of the hip joint. The labrum has been shown to increase by an average of 22% to the articulating surface area of the hip joint. The labrum lowers the stress on the articular cartilage by regulating fluid transport between the central and peripheral compartments and by distributing contact pressure across the articulating surfaces. This fluid seal is one of the most important function of the labrum, as it produces a negative intra-articular pressure, significantly increasing joint stability.

If the shape of the femoral head and acetabulum do not perfectly match, femoral-acetabular impingement can occur. This is a genetic condition that predisposes the hip labrum to traumatic injury. Hip labral tears are associated with certain activities such as gymnastics, running, jumping, tennis, soccer, and cycling. The diagnosis is suspected when hip ROM, especially forced flexion with internal rotation duplicates the groin pain the patient typically experiences. The diagnosis is confirmed with MRI scanning.

There is a paucity of non-operative treatments for hip labral tears. The most common and effective solution includes decreasing or avoiding activities that causes pain. Physical therapy has shown little efficacy. Surgical options are typically performed arthroscopically. The surgical options include either repairing the labral tear or, more commonly, resecting the torn labrum. Krych, et al, reported the results of hip arthroscopic treatment for labral tears. They reported 59 patients, the mean age was 46, and the mean follow up was 5 years. They reported a clinical failure rate of 45% due to repeat surgery in 20% and continued pain in 25%.

Published reports of using injections of autogenous bone marrow concentrate (BMC) with mesenchymal stem cells (MSC) to treat shoulder labral tears indicate safety and efficacy. The MSC is the only cell that can differentiate into a chondroblast, osteoblast, or fibroblast. It has historically been thought differentiation was the mechanism by which MSC injections treated joint pathology. It has become increasingly recognized the efficacy of using MSC injections to treat various conditions is due to their paracrine function of releasing anti-inflammatory growth factors (GF) and exosomes. These GFs and exosomes can be injected without any living cells. This case report, will introduce the concept of using acellular bone marrow-derived MSC GEs and exosomes to treat hip labral tears. This is described as an extracellular vesicle isolate product (EVIP). The paper will discuss the rationale of why acellular will replace all current cellular biologic therapies, both autogenous and allogeneic for the treatment of various joint pathologies.

### a) Materials and Methods

This is a case report of an EVIP injection for the treatment of a hip labral tear. A hip labral tear is characterized by a locking, clicking, or catching in the hip joint. Pain is felt in the groin or radiating into the trochanteric area. Patients complain of stiffness or limited hip range of motion. MRI scanning is used to diagnose a labral tear of the hip joint. The patient is a 50-year old, extremely active triathlete, personal trainer, and health instructor, Following several months of untreated groin pain, she presented on Jun. 11, 2019 with an increasing right groin and buttocks pain radiating into the upper leg. Her symptoms were exacerbated with activities requiring hip ROM. MRI scanning of the hip joint on Jun. 4, 2019 revealed a superior labrum tear, superior anterior labral fraying, tear of the gluteus minimus tendon, trochanteric bursal inflammation, and common hamstring tendinosis. The hip joint did not have osteoarthritis. She had been treated with NSAIDs, weekly active release, and chiropractic treatments. On physical examination she had an overall 20% loss of hip ROM. Forced internally rotation and flexion replicated and severely exacerbated her pain. The patient felt overall her right hip was less than 50% normal compared to the opposite hip. The patient was evaluated with a Brief Pain Inventory (BPI), a Lower Extremity Functional Index (LEFI), and an Oswestry Disability Index (ODI) prior to the injection and at follow-up.

### (1) The Hip Injection

After counseling and consent, the patient on Jun. 11, 2019 underwent the hip injection. The right groin and buttocks area were sterilized with a betadine skin prep. A 20-gauge needle was placed through an anterior approach into the hip joint. Needle placement was verified by fluoroscopy. At this point, 2 cc of the frozen EVIP (ExoFlo-Direct Biologics, St. Louis MO) was thawed to room temperature and placed into the joint.

### b) Clinical Results

The patient was put on restricted physical activity for I week following the procedure. Passive low-resistance range of motion was encouraged immediately. The patient returned to full activities at 10 days. Six weeks post-procedure, she was able in one day to run 7 miles, kayak 2 hours and swim 600 yards. She was able to sleep like a “rock” and wake up with no pain. The patient opines she has experienced an overall 75% improvement from her preprocedure hiplabral tear symptoms.

### (1) Follow Up MRI Scan

A follow-up gadolinium arthrogram MRI scan of the right hip was performed on Aug. 28, 2019. The scan showed NO discrete labral tear. There was no evidence of tendinosis or interstitial tearing of the gluteus minimums tendon. There was no trochanteric bursal inflammation. The impression was a negative MRI of the right hip joint. Her clinical results from pre-procedure to 12-week FU are shown in both (FIG. 1) and (Table 1). They detail the improvement noted in her Brief Pain Inventory (a lower score is better), Oswestry Disability Index (a lower score is better), and her Lower Extremity Functional Score (a higher score is better).

TABLE 1
Physical evaluation measurements with the percent
improvement over the initial 12 weeks.

| | | | | Percent |
|---|---|---|---|---|
| | Baseline | 6 Weeks | 12 Weeks | Improvement |
| BPI | 64 | 14 | 10 | 84% |
| ODI | 23 | 11 | 9 | 61% |
| LEFS | 31 | 48 | 51 | 61% |

1.5

### c) Discussion

The patient is an active 50 YO triathlete with a history of increasing pain from a documented hip labral tear. She underwent a single anterior hip injection of 2 cc of bone marrow-derived mesenchymal stem cell EVIP (ExoFlo-Direct Biologics, St. Louis MO) containing active growth factors and exosomes. Two weeks later, her labral tear symptoms were improved by 75%. This improvement has been maintained through three months. Her pre-injection arthrogram MRI documents a superior labrum tear, superior anterior labral fraying, tear of the gluteus minimus tendon, trochanteric bursal inflammation, and common hamstring tendinosis. The hip joint did not have osteoarthritis. Her post-injection arthrogram MRI documents no evidence of a labral tear. There was no evidence of tendinosis or interstitial tearing of the gluteus minimus tendon. There was no trochanteric bursal inflammation. The impression was a negative MRI of the right hip joint. She will continue to be closely monitored for two years.

The non-operative treatment for symptomatic labral tears is basically to avoid activities that cause pain. The surgical options are either arthroscopic removal or repair of the labrum. Published results indicate a 45% failure rate with arthroscopic hip labral surgery.

Based on understanding the biology of labral tears, non-operative treatment will be an injection of acellular MSC derived growth factors and especially exosomes. The exosome is a tiny 30 to 150 nanometer-sized (1 billionth of a meter) bi-phospholipid membrane-enclosed structure created by the endosomes. An MSC (12 to 18 microns) is 1,000 times larger than an exosome. The diameter of a hair is 80,000 nanometers. Exosomes contain growth factors, signaling lipids and micro, and messenger RNA. These paracrine factors can be placed into any joint in concentrations of 100,000 or more times that of any cellular MSC treatment. These growth factor proteins and exosomes function in a paracrine fashion to both, directly and indirectly, alter the inflammatory environment of a painful labral tear back to a normal non-painful physiologic environment and stimulate fibrocartilaginous healing.

The future acellular treatment for labral tears will involve a two-step process: first, highly concentrated anti-inflammatory bone marrow-derived MSC growth factors and exosomes are injected into the painful joint. Second, these growth factors and exosomes enter the recipient, synovial MSCs to stimulate the production of new chondroblasts and anti-inflammatory secretomes, chemokines, and cytokines. These effects have been shown to heal fibrocartilaginous tears. This acellular biologic treatment can all be achieved with a single joint injection. The future of regenerative medicine in orthopedics and spine may well be the utilization of highly concentrated acellular MSC derived growth factors and especially exosomes.

### 2. Example 2: Intra-Articular Injection of an Extracellular Vesicle Isolate to Treat Shoulder Osteoarthritis in an Athlete

Shoulder osteoarthritis (OA) has been demonstrated in cadaver and radiographic studies to affect up to 339% of patients over the age of 60. Patients that present with shoulder OA have pain, crepitus, loss of motion and decreased ability to place their hand at a desired point in space. Limiting the ability to place your hand where you desire severely impairs activities of daily living. The nonsurgical treatments for shoulder OA, in an attempt to maintain range of motion, include shoulder exercises, the use of analgesics and non-steroidal anti-inflammatory medications. If these non-surgical treatments fail to relieve the OA impairment, then the default surgical treatment is total shoulder arthroplasty (TSA).

Recently it has become increasingly understood by researchers and physicians that the clinical efficacy of utilizing mesenchymal stem cells (MSCs) to treat osteoarthritis (OA). Additionally, it is becoming evident that the relief is not dependent on the cells differentiating into articular cartilage but entirely on their paracrine release of growth factors (GFs) and exosomes, with the cells possibly interfering with the healing process. Active growth factors and exosomes can function without the cells when delivered into an arthritic joint. This is a case report of an acellular MSC derived Extracellular Vesicle Isolate Product (EVIP) injection for the treatment of shoulder osteoarthritis. Shoulder OA is defined by pain and stiffness in the shoulder joint, worsened by exercise and decreased shoulder range of motion. This case report will introduce the concept of using EVIP containing active growth factors and exosomes to treat OA and the rationale of why acellular biologic treatments could replace current autogenous and allogeneic cellular biologic therapies.

### a) Methods

The patient, a 57-year-old male athlete that is right hand dominant, presented with a greater than 14-year history of increasing bilateral shoulder pain right-side much worse than left side. Pre-procedure radiographs and MRI scanning were used to distinguish osteoarthritis of the glenohumeral joint from rotator cuff abnormalities. A previous MRI scan of the shoulder obtained on Jan. 5, 2006 (13 years before this procedure) revealed a Kellgren-Lawrence score of 4, which is bone-on-bone osteoarthritis of the glenohumeral joint.

The patient was further evaluated at each visit with a Physical examination of the shoulder and using QuickDASH (QD), a measure of shoulder function with lower scores being ideal; The Upper Extremity Functional Index (UEFI) with higher percentages being better scores; Brief Pain Inventory (BPI) with higher scores indicate higher pain, as well as a patient self-rated evaluation of overall shoulder improvement. These physical evaluation procedures were performed at three different clinic visits: Pre-procedure, and then at six weeks and twelve weeks post-procedure. On initial physical examination, there was observed an average 75% loss of internal, external and abduction of the right glenohumeral joint. All range of motion evaluations were associated with severe pain, crepitus and bone on bone clunking. Pre-procedural radiograph of the shoulder is shown in FIG. 2.

### (1) Shoulder Injection

After counseling and consent the patient's right shoulder was prepared with betadine skin solution. A 20-gauge needle was placed through an anterior approach into the glenohumeral joint. The needle was placed against the humoral head within the shoulder joint capsule. Needle placement was verified by internal and external rotation of the humeral head causing the needle to move. A 2.0 cc preparation of the frozen EVIP (ExoFlo-Direct Biologics, St. Louis, MO) was thawed to room temperature and placed into the gleno humeral joint.

The patient was placed on restricted physical activity for 1 week following the procedure. Passive low-resistance range of motion was encouraged immediately. Two weeks after the procedure the patient returned to fall activities.

### b) Results

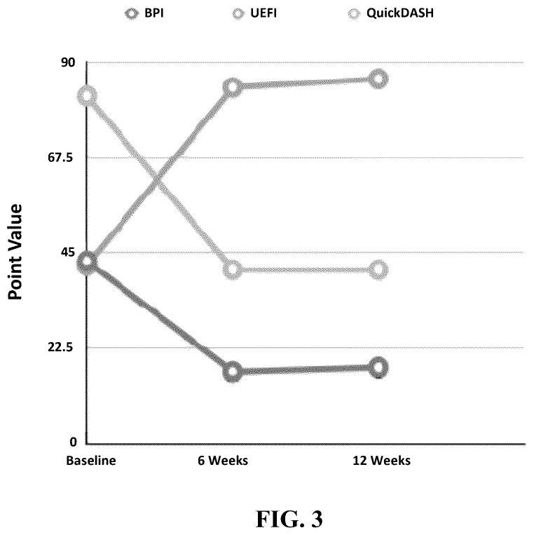

The patient's self-rated overall shoulder improvement was 75% from 2 weeks after the injection to the 12-week evaluation. The QuickDASH, The Upper Extremity Functional Index, and Brief Pain Inventory were all improved within 2 weeks and that improvement was maintained out to 12 weeks as shown in the graph in FIG. 3 and the values displayed in Table 2.

TABLE 2
Physical evaluation measurements with the percent
improvement over the initial 12 weeks.

| | | | | Percent |
|---|---|---|---|---|
| | Baseline | 6 Weeks | 12 Weeks | Improvement |
| BPI | 43 | 17 | 18 | 58% |
| UEFI | 42 | 84 | 86 | 51% |
| QuickDASH | 82 | 41 | 41 | 50% |

Overall shoulder improvement was self-rated to be 75%. Post-procedure glenohumeral range of motion improved by 33% based on physical examination.

### e) Discussion

The patient is a male 57-year-old active athlete that presented with a greater than 14-year history of increasing right dominant shoulder pain. He was diagnosed with Kellgren-Lawrence grade four bone on bone glenohumeral osteoarthritis without rotator cuff abnormalities. It is not anticipated to observe regeneration of the articular cartilage. Follow up x-rays will not change. He underwent a single injection of 2 cc of a bone marrow derived EVIP containing active growth factors and exosomes. The injection was performed without complications. Within two weeks the patient was 50% improved or more based on the BPI, UEFI and QD measurement scales. He rated his shoulder to overall be 75% improved. He had returned to full activities by two weeks. These results have been maintained out to 12 weeks. The patient will continue to be monitored at later timepoints. The pain relief observed is felt to be due to the decrease in inflammation of the synovial capsule.

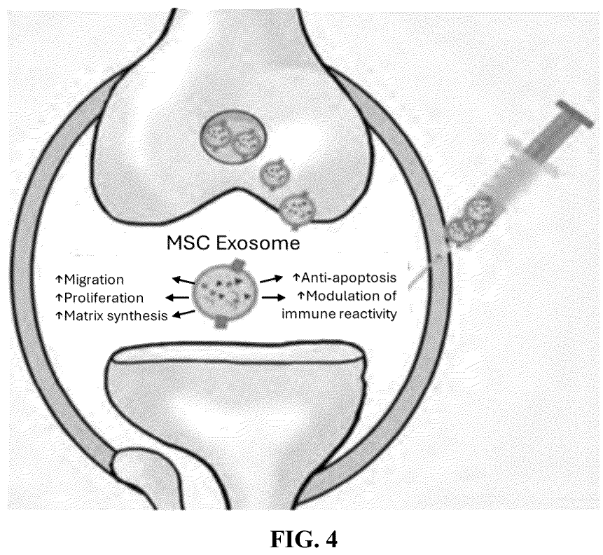

The shoulder is a di-arthrodial joint with a synovial lining and a joint capsule. The synovial capsule contains numerous synovial MSCs (more than found in bone marrow or adipose). These MSCs have more chondrogenic potential than bone or adipose MSCs. During the development of OA, pro-inflammatory and catabolic growth factors are produced by these synovial MSCs. This creates a chronically inflamed painful and degenerative joint environment. Bone marrow concentrate (BMC) contains on average only about 2.500 MSCs per cc. Despite the incredibly small number of MSCs found in BMC; there is extensive literature reporting clinical efficacy in the use of BMC for the treatment of OA. This effect cannot be dependent upon BMC/MSC cell survival or differentiation. The efficacious effect must be from the release of acellular paracrine factors. The future of the biologic treatment of OA will be the utilization of acellular MSC derived growth factors and especially exosomes. The exosome is a tiny 30 to 150 nanometersized (1 billionth of a meter) bi-phospholipid membrane enclosed structure created inside the cell's cytoplasm. An MSC (12 to 30 microns) is ˜1,000 times larger than an exosome. For reference, the diameter of a hair is 80,000 nanometers or 80 microns. Exosomes contain growth factors, signaling lipids and micro, and messenger RNA (miRNA and mRNA), The RNA contents in exosomes mediate most of their anti-inflammatory effects. The RNA is packaged into an exosome along with numerous growth factors naturally by the cell. These cell-derived paracrine factors can be placed into any joint in concentrations of 100,000 or more times that of any cellular MSC treatment and function in a paracrine fashion to, directly and indirectly, alter the inflammatory environment of any painful arthritic joint back to a normal non-painful homeostatic physiologic environment. FIG. 4 illustrates this process.

The future acellular treatment for OA will involve a two-front attack. First, highly concentrated anti-inflammatory MSC derived growth factors are injected into the arthritic joint. These growth factors will enter the nucleus of the recipient synovial MSC. The EVIP growth factors will stimulate transcription of mRNA containing cellular instructions to produce continuous anti-inflammatory and regenerative secretomes, chemokines, and cytokines. These are released from the recipient cells into the synovial fluid. Second, the highly concentrated exosomes from the EVIP will enter recipient cells to deliver their mRNA and miRNA. This delivered mRNA will directly undergo translation in the recipient synovial MSC ribosomes to produce an anti-inflammatory and regenerative secretome (growth factors, cytokines, chemokines and extracellular vesicles). These salubrious effects could last months or years. This acellular biologic treatment can all be achieved with a single arthritic joint injection and avoids the morbidity and cost of obtaining autogenous MSCs. The future of regenerative medicine in orthopedics and spine may well be the utilization of highly concentrated acellular MSC bone marrow derived growth factors and especially exosomes.

### 3. Example 3: Intra-Articular Injection of an Extracellular Vesicle Isolate Product to Treat Hip Osteoarthritis

Hip osteoarthritis (OA) has demonstrated, in both cadaver and radiographic studies, to affect up to 55 million patients over the age of 60. Patients with hip OA have pain, crepitus, loss of motion, and decreased ability to weight bear or ambulate. Limiting the ability to ambulate severely impairs activities of daily living. The nonsurgical treatments forhip OA according to the American Academy of Orthopedic Surgeons (AAOS), include weight loss, gentle exercise, and the use of non-steroidal anti-inflammatory medications. The surgical treatment forhip OA is total hip arthroplasty (THA). The AAOS does not recommend hip arthroscopy or the use of any Hyaluronic Acid injections.

Over the last few years, it has become increasingly understood by researchers and clinicians that the clinical efficacy of utilizing mesenchymal stem cells (MSCs) to treat osteoarthritis (OA) is not dependent on the cells differentiating into articular cartilage but entirely on their paracrine release of growth factors (GFs) and exosomes. Living MSCs are not required to accomplish the release of GEs and exosomes into an arthritic joint. This case report will introduce the concept of using an acellular MSC derived extracellular vesicle isolate product (EVIP) containing active growth factors and exosomes to treat hip OA as well as the rationale of why acellular may replace all current cellular biologic therapies both autogenous and allogeneic presently in use.

### a) Materials and Methods

This is a case report of an EVIP injection for the treatment of hip osteoarthritis. OA is defined by swelling, pain, and stiffness in the hip joint. Symptoms are typically worsened by weightbearing and ambulation. Radiographs and MRI scanning were used to grade osteoarthritis of the hip joint from one to four using the Kellgren-Lawrence scale.

The patient is a 63-year-old retired Chicago Fireman. He presented with increasing pain in the left groin and a progressive loss of ability to continue his daily health club fitness routine. He experienced a progressive loss of hip mobility. MRI scanning and radiographs of the left hip joint were compatible with Kellgren-Lawrence Grade 3 osteoarthritic changes of the left hip joint. On physical examination, he had an antalgic limp and a positive Trendelenburg sign. Passive ROM of the hip joint was associated with the reproduction of severe groin pain, crepitus, and a loss of internal rotation. The patient had a BMI of 27. NSAIDs had failed to provide adequate pain relief. The patient was seriously considering total hip arthroplasty. In an attempt to avoid surgery, he elected to have an injection of an EVIP containing active GFs and exosomes into his hip.

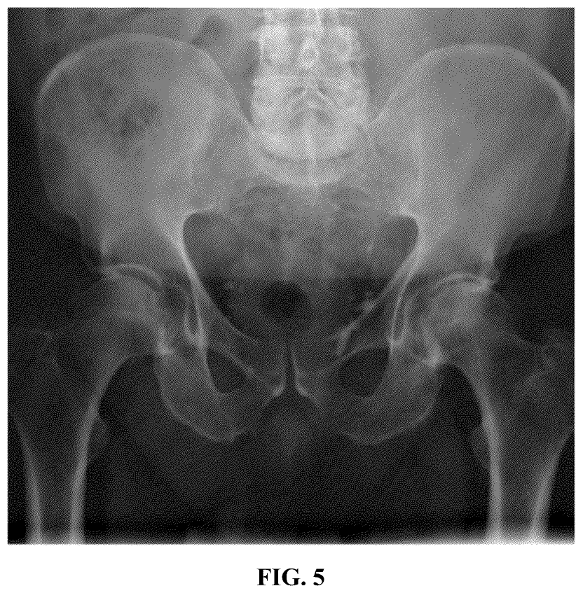

Radiographs of a pelvis are shown in FIG. 5. This x-ray shows a normal right hip and grade three OA hip on the left. The left hip was sterilized with betadine skin prep. A 20-gauge spinal needle was placed through an anterolateral approach into the hip joint. Needle placement was verified by fluoroscopy. At this point, 2 cc of the frozen EVIP (ExoFlo-Direct Biologics, St. Louis MO) was thawed to room temperature and placed into the joint. The patient was put on restricted physical activity for one week following the procedure. Passive low-resistance range of motion was encouraged immediately. The patient returned to full activities at two weeks.

### Clinical Results

At the three-month follow-up, the patient had returned to his previous fitness routine without limitation. He is no longer limited in his exercise profile and has enjoyed the return of a functional pain-free hip ROM. He has returned to full activities without restrictions.

### b) Discussion

The hip is a di-arthrodial joint with a synovial lining and a joint capsule. The synovial capsule contains numerous synovial MSCs (more than those found in bone marrow or adipose tissue). These MSCs have more chondrogenic potential than bone or adipose MSCs. During the development of OA, proinflammatory growth factors are produced by these synovial MSCs. This creates a chronically inflamed painful joint environment. Bone marrow concentrate (BMC) contains on average only about 2,500 MSCs per cc. Despite the incredibly small number of MSCs found in BMC; there is an extensive amount of literature reporting the clinical efficacy in animals and humans using BMC for the treatment of OA. This effect cannot be dependent upon BMC/MSC cell survival or differentiation. This efficaciousness must be from the release of acellular paracrine factors. The future of the biologic treatment of OA will be the utilization of acellular MSC derived growth factors and especially exosomes.

The exosome is a tiny 30 to 150 nanometer-sized (1 billionth of a meter) bi-phospholipid membrane-enclosed structure created by the Golgi body or apparatus. An MSC (12 to 18 microns) is 1,000 times larger than an exosome. The diameter of a hair is 80,000 nanometers. Exosomes contain growth factors, signaling lipids, and micro and messenger RNA. The RNA contents within exosomes mediate most of their anti-inflammatory effects. The RNA is placed into an exosome along with numerous peptide growth factors. These paracrine factors can be placed into any joint in concentrations of 100,000 or more times that of any cellular MSC treatment. These growth factor proteins and exosomes will function in a paracrine fashion to both directly and indirectly, alter the inflammatory environment of any painful arthritic joint back to a normal non-painful physiologic environment.

The future acellular treatment for OA will involve a two-front attack. First, highly concentrated anti-inflammatory MSC derived growth factors are injected into an arthritic joint. These growth factors will enter the nucleus of the recipient synovial MSC. The EVIP growth factors will stimulate transcription of mRNA containing instructions for the production of continuous anti-inflammatory secretomes, chemokines, and cytokines. These will be released from the recipient synovial MSC into the synovial fluid. Second, the highly concentrated exosomes from the EVIP will enter recipient synovial MSCs to deliver their mRNA. This mRNA will undergo direct translation in the recipient synovial MSC ribosomes to produce anti-inflammatory secretomes, cytokines, and chemokines.

These salubrious effects could last months or years. This acellular biologic treatment can all be achieved with a single arthritic joint injection, without requiring the morbidity and cost of obtaining autogenous MSCs. The future of regenerative medicine in orthopedics and spine may well be the utilization of highly concentrated acellular MSC derived growth factors and especially exosomes.

### 4. Example 4: Treatment of Elbow Arthritis with An Extracellular Vesicle Isolate Intra-Articular Injection

Primary osteoarthritis (OA) of the elbow is an uncommon condition associated with a genetic predisposition. This occurs predominantly within the ulno-humeral joint of the dominant extremity of patients who engage in heavy sport or labor. Posttraumatic elbow OA is by far more common. A variety of traumatic insults may ultimately result in specific forms of posttraumatic OA to the elbow. The most common complaints of patients with either primary or posttraumatic arthritis of the elbow are pain and/or loss of motion. Loss of extension and pronation can be compensated more easily than loss of flexion and supination.

Nonoperative management remains the mainstay of initial treatment for elbow OA. This typically includes elbow sleeves, nonsteroidal anti-inflammatory medications, and intra-articular corticosteroid injections. Avoidance of aggressive terminal flexion and extension activities can result in substantial relief of pain (weight lifting, boxing, etc.). A course of supervised rehabilitation by a certified therapist is reserved typically for patients presenting with an acute-on-chronic presentation of symptoms with an associated effusion and limitations in motion.

Surgical management is indicated for patients with severe elbow pain or significant loss of mobility with resultant impairment of upper extremity function and limitation with daily activities. Advances in elbow arthroscopy have resulted in favorable outcomes and have totally replaced any open surgical debridement. Arthroscopic debridement of the elbow, particularly in a younger patient population, bas reasonable results with improvements in pain and range of motion. It is important to note the published procedures are performed by surgeons with substantial experience with safe, meticulous techniques in elbow arthroscopy. Total elbow arthroplasty is most appropriate for the low-demand, elderly patient (>60 year old) with inflammatory, posttraumatic, or primary elbow arthritis. Total elbow arthroplasty is rarely applied in the setting of elbow OA in a younger and typically male population.

There is a huge void between non-operative and operative treatment of elbow OA. This is a case report of a young very active Chiropractor with OA of the elbow. She was treated with a single intra-articular injection of a bone marrow derived mesenchymal stem cell (MSC) extracellular vesicle isolate containing active growth factors and exosomes.

### a) Materials and Methods

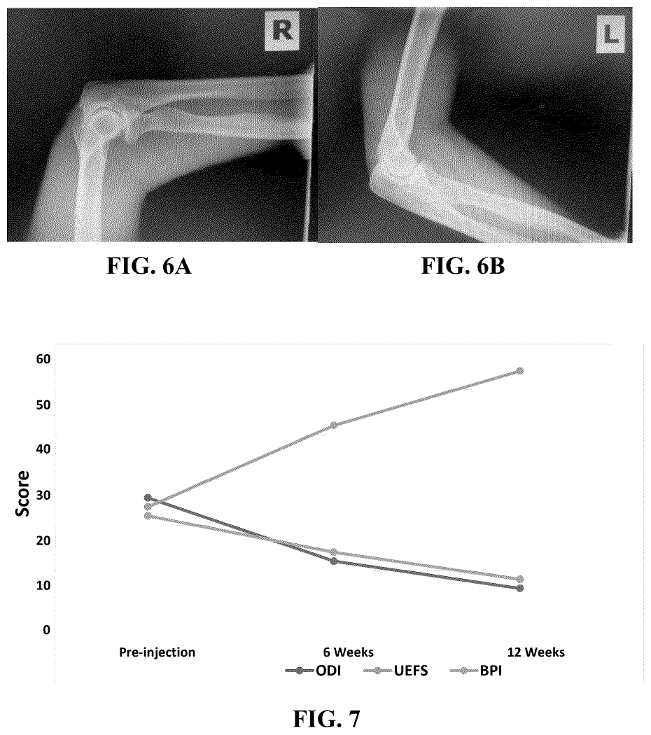

The patient is a very active healthy 60-year old practicing Chiropractor. She initially injured her dominant right elbow at the age of 16 in a severe water-skiing accident. She suffered a complete dislocation of the elbow with her ulna and radius located posterior to the humerus. There was no neurovascular injury. The elbow was reduced under general anesthesia. After recovery she lacked 150 of full extension and 100 of full flexion. There was no reduction in supination or pronation. She suffered a second injury at the age of 58 resulting in a complete radial head dislocation. Following this injury, she developed traumatic arthritis of the radial capitellum joint with daily pain and joint swelling exacerbated with activities. Her ability to practice chiropractic care was severely limited. She regularly took NSAIDs and ice therapy after work. Her physical examination of the elbow revealed a 150 loss of elbow extension and 100 loss of flexion. She had a 10° loss of pronation and supination. There was one plus swelling of the elbow joint. Her radiographs reveal grade three Kellgren-Lawrence osteoarthritis of the elbow joint (FIGS. 6A and 6B).

### (1) Elbow Injection

After counseling and consent, the patient on Jun. 11, 2019 underwent an elbow injection. The right elbow joint area was sterilized with a betadine skin prep. A 20-gauge needle was placed through a lateral and medial approach into the elbow joint. At this point 3 cc of the frozen EVIP (ExoFlo-Direct Biologics, St. Louis MO) was thawed to room temperature and placed into the medial and lateral side of the elbow joint. The patient experienced no adverse reactions. The entire procedure took 15 minutes.

### b) Results

Following the injection, the elbow joint became painful and swollen for several days. She was unable to work for a week. Her symptoms improved over the next 4 weeks. By 6weeks post injection she felt her elbow was 50% better. This improvement has continued through 3 months and the elbow is now 70% improved from pre-injection. Elbow range of motion has not changed following the EVIP injection (Table 3 and FIG. 7).

TABLE 3

| | Pre-injection | 6 Weeks | 3 months | Percent Improvement |
|---|---|---|---|---|
| ODI | 26 | 12 | 6 | 77% |
| UEFS | 24 | 42 | 54 | 56% |
| BPI | 22 | 14 | 8 | 64% |

### c) Discussion

This is a case study of a 60-year old active practicing chiropractor with a many year history of an increasingly symptomatic traumatic osteoarthritis dominant elbow. She suffered a severe posterior elbow dislocation at the age of 16. Her symptoms were greatly exacerbated by work to the point she was considering retirement. This was a major decision because she was in a solo practice with financial overhead. Her surgical option was total elbow arthroplasty. She would be unable to return to her chiropractic practice following this surgical procedure. The goal of the surgery was to attempt to improve her activities of daily living not a Return to strenuous work. In an attempt to avoid surgery and continue to practice chiropractic she elected to try an elbow injection of the bone marrow derived MSC EVIP containing active growth factors and exosomes (ExoFlo-Direct Biologics, St. Louis MO). Three months following the injection her elbow is 70% improved and she is working full time.

The elbow is a di-arthrodial joint with a synovial lining and a joint capsule. The synovial capsule contains numerous synovial MSCs (more than found in bone marrow or adipose). These MSCs have more chondrogenic potential than bone or adipose MSCs. During the development of OA, pro-inflammatory growth factors are produced by these synovial MSCs. This creates a chronically inflamed painful joint environment. Bone marrow concentrate (BMC) contains on average only about 2,500 MSCs per cc. Despite the incredibly small number of MSCs found in BMC; there is extensive literature reporting clinical efficacy in animals and humans using BMC for the treatment of OA. This effect cannot be dependent upon BMC/MSC cell survival or differentiation. The efficacious effect must be from the release of acellular paracrine factors. The future of the biologic treatment of OA will be the utilization of acellular MSC derived growth factors and especially exosomes. The exosome is a tiny 30 to 150 nanometer-sized (1 billionth of a meter) bi-phospholipid membrane-enclosed structure created by the Golgi body or apparatus. An MSC (12 to 18 microns) is 1,000 times larger than an exosome. The diameter of a hair is 80,000 nanometers. Exosomes contain growth factors, signaling lipids and micro, and messenger RNA. The RNA contents in exosomes mediate most of their anti-inflammatory effects. The RNA is placed into an exosome along with numerous peptide growth factors. These paracrine factors can be placed into any joint in concentrations of 100,000 or more times that of any cellular MSC treatment. These growth factor proteins and exosomes will function in a paracrine fashion to, directly and indirectly, alter the inflammatory environment of any painful arthritic joint back to a normal non-painful physiologic environment.

The future acellular treatment for OA will involve a two-front attack. First, highly concentrated anti-inflammatory MSC derived growth factors are injected into the arthritic joint. These growth factors will enter the nucleus of the recipient synovial MSC. The EVIP growth factors will stimulate transcription of mRNA containing instructions for the production of continuous anti-inflammatory secretomes, chemokines, and cytokines. These will be released from the recipient synovial MSC into the synovial fluid. Second, the highly concentrated exosomes from the EVIP will enter recipient synovial MSCs to deliver their mRNA. This delivered mRNA will directly undergo translation in the recipient synovial MSC ribosomes to produce anti-inflammatory secretomes, cytokines, and chemokines. These salubrious effects could last months or years. This acellular biologic treatment can all be achieved with a single arthritic joint injection, not requiring the morbidity and cost of obtaining autogenous MSCs. The future of regenerative medicine in orthopedics and spine may well be the utilization of highly concentrated acellular MSC bone marrow derived growth factors and especially exosomes.

### 5. Example 5: Osteoarthritis Treatment by EVIP Administration

Extracellular Vesicle Isolate Product (EVIP) derived from bone marrow mesenchymal stem cells contains active Growth Factors (over 800) and Exosomes (over 10 Billion per cc). This is the first IRB sponsored report on the safety and efficacy of an EVIP injection to treat OA. Thirty-three former Navy Seals were injected with the EVIP for knee (n=58), shoulder (n=32), elbow (n=16), hip (n=12), ankle (n=8) or wrist (n=6) OA. At three-month follow-up, the average knee patient improved 70% in Brief Pain Inventory (BPI), 67% in Oswestry Disability Index (ODI) and 62% in Lower Extremity Functional Scale (LEFS), the average shoulder patient improved 68% in BPI, 82% in ODI, 74% in QD and 68% in UEFS, the average elbow patient improved 76% in BPI 81% in QD and 76% in UEPS, the average hip patient improved 70% in BPI, 72% in ODI and 64% in LEFS, the average ankle patient improved 70% in BPI, 72% in ODI and 64% in LEFS, and the average wrist patient improved 68% in BPI, 64% in QD and 74% in UEFS. All improvements were to a p<0.001. There were no complications and no patient was made worse from the EVIP injection. At 3-month follow-up the EVIP injection for OA appears sale and efficacious and should be considered prior to joint replacement.

### a) EVIP Characterization

The product is derived from the bone marrow of a 22 year-old female donor whose MSCs have been master banked and registered with the FDA. The end product from these cells contains over 800 different active growth factors and well over 30 billion exosomes per cc. The product is used in a frozen form. Sterilization is achieved through ultrafiltration, not radiation. Acellular exosomes, derived from bone marrow MSCs, provide a consistent product with extensive characterization which includes advanced particle analysis, proteomic evaluation, and USP<71> sterility assurance. Cytokine and growth factor identification and quantification are also performed. Think of acellular exosomes as a therapeutic quality product that is consistent, standardized, and quality tested regarding dose and activity.

### (1) Patient Demographics

The number of patients undergoing treatment for knee, shoulder, elbow, hip, ankle and wrist along with average BMI and average age are shown in Table 4.

Injection Technique: All the injected joints were sterilized with betadine skin prep. Under fluoroscopy a 20-gauge needle was placed into the arthritic joint. Needle placement was verified under fluoroscopic control. At this point, 2 cc of the frozen EVIP (ExoFlo-Direct Biologics, St. Louis MO) was thawed to room temperature and placed into the joint. The entire procedure of injecting four joints per patient required 30 minutes on average.

Patients were not prescribed any pain medications. They were put on a restricted physical activity for 2 weeks following the procedure. Passive low-resistance range of motion was encouraged immediately. After two weeks, patients were allowed to return to full activity.

### (2) Statistical Tests

Univariable data comparisons were analyzed by two-tailed Student's t-test with a 95% confidence interval (α=0.05. Microsoft Excel). Multivariable data were evaluated by analysis of variance (ANOVA) using JMP 9 statistical analysis software (SAS Institute, Cary NC).

### b) Results

Every patient was contacted 12 hours, 24 hours, 48 hours and at 2 weeks, 6 weeks and 3 months to discuss any and all side effects from the EVIP injection. There were no adverse-affects reported by any patient from the EVIP injection. No patient was made clinically worse from the EVIP injection.

TABLE 4
Description of Patient Demographics

| | Shoulder | Hip | Knee |
|---|---|---|---|
| Number of patients | 32 | 12 | 58 |

BMI=Average 28.5, Range=23-35.

Average age=48.8 years, Range=36-70.

Knee=58, Shoulder=32, Elbow=16, Hips=12, Ankles=8, Wrist=6, Total number of joints injected=132.

### E. References

Ball C M, Meunier M, Galatz L M, Calfee R, Yamaguchi K. Arthroscopic treatment of post-traumatic elbow contracture. Journal of Shoulder and Elbow Surgery. 2002; 11(6): 624-629.
Beitzel K, Solovyova O, Cote M P, Apostolakos J, Russell R P, McCarthy M B, et al. The future Role of Mesenchymal Stem Cells in the Management of shoulder Disorders. Arthroscopy. 2013; 29(10): 1702-11.
Biswas D. Wysocki R W, Cohen M S: Primary and Secondary Arthritis of the Elbow. Arthritis. 2013, May 27.
Black L L, Gaynor J, Adams C, Dhupa S, Sams A E, Taylor R. et al. Effect of intraarticular injection of autologous adipose-derived mesenchymal stem and regenerative cells on clinical signs of chronic osteoarthritis of the elbow joint in dogs. Vet Ther. 2008; 9: 192-200.
Black L L., Gaynor J, Gahring D, et al. Effect of adipose-derived mesenchymal stem and regenerative cells on lameness in dogs with chronic osteoarthritis of the coxofemoral joints: a randomized, double-blinded, multicenter, controlled trial. Vet Ther 2007; 8: 272-84.
Caplan A I, Correa D. The MSC: An injury drugstore. Cell Stem Cell. 2011 Jul. 8; 9(1): 11-5.
Caplan A I, Dennis J E. Mesenchymal stem cells as trophic mediators. J Cell Biochem 2006; 98: 1076-1084.
Centers for Disease Control and Prevention (CDC) Prevalence and most common causes of disability among adults—United States, 2005. Morbidity and Mortality Weekly Report 2009; 58(16): 421-426.
Chang C H, Huo T F, Lin F H, et al. Tissue engineering based cartilage repair with mesenchymal stem cells in a porcine model. J Orthop Res 2011; 29: 1874-80.
Chang Y, Wu K, Harn H. Exosomes and Stem Cells in Degenerative Disease Diagnosis and Therapy. Cell Transplantation, Apr. 25, 2018.
Cheng L, Zhang K, Wo S, Cui M, Xu T. Focus on Mesenchymal Stem Cell-Derived Exosomes: opportunities and Challenges in Cell-Free Therapy. Stem Cells Int. 2017; 2017: 6305295.
Chew E. Prakash R, Khan W, Mesenchymal Stem Cells in Human Meniscal Regeneration: A Systemic Review. Ann Med Surg. 2017; 24: 3-7.
Dwyer M K I, Jones H L, Hogan M G, Field R E, McCarthy J C, Noble P C. The acetabular labrum regulates fluid circulation of the hip joint during functional activities. Am J Sports Med. 2014; 42(4): 812-9.
Fan J., Varshney RR, Ren L., Wang D A. Synovium-Derived Mesenchymal stem cells: A new source for musculoskeletal regeneration. Tissue Engineering Part B Review 2009 March; 15(1);75-86.
Feng G et al. Transplantation of mesenchymal stem cells and nucleus pulposus cells in a degenerative disc model in rabbits: a comparison of 2 cell types as potential candidates for disc regeneration. J Neurosurgery Spine 2011; 14: 322-9.
Freitag J, Bates D. Mesenchymal stem cell therapy in the treatment of Osteoarthritis: reparative pathways, safety, and efficacy: A Review. BMC Musculoskeletal Disorders. 2016; 17: 230.
Frisbie D D, Smith R K W (2010) Clinical update on the use of mesenchymal stem cells in equine orthopaedics. Equine Veterinary Journal, 42: 86-9.
Guerico A, Di Marco P, Casella S, et al. Production of canine mesenchymal stem cells from adipose tissue and their application in dogs with chronic osteoarthritis of the humeroradial joints. Cell Biol Int 2012; 36: 189-94.
Harris J D. Hip labral repair: options and outcomes. Curr Rev Musculoskelet Med. 2016; 9(4): 361-367.
Hiyama A, Mochida J, Iwashina T, Omi H, Watanabe T, Serigano K, Tamura F, Sakai D. Transplantation of mesenchymal stem cells in a canine disc degeneration model. J Orthop Res 2008; 26: 589-600.
Kellgren J, Lawrence J. Radiological assessment of Osteo-Arthrosis. Ann Rheum Dis December 1957; 16(4): 494-502.
Kelly E W, Bryce R, Coghlan J, Bell S. Arthroscopic debridement without radial head excision of the osteoarthritic elbow. Arthroscopy. 2007; 23(2): 151-156.
Koga H., Muneta T., et al. Synovial Stem cells are Regionally Specified According to Local Microenvironments after Implantation for Cartilage Regeneration. Stem Cells 2007; 25: 689-96.
Krych A J, Kuzma S A, Kovachevich R, Hudgens J L, Levy B A. Modest Mid-term outcomes after Isolated Arthroscopic Debridement of Acetabular Tears. Knee Surg Sports Traumatol Arthrosc. 2014; 22(4): 763-7.
Lee K B, Hui J H, Song I C, et al. Injectable mesenchymal stem cell therapy for large cartilage defects—a porcine model. Stem Cells 2007; 25: 2964-71.
Li Z, Wang Y, Xiao K, Weng X. Emerging Role of Exosomes in the Joint Diseases, Cell Physiol Biochem. 2018; 47(5): 2008-2017.
Little C P, Graham A J, Carr A J. Total elbow arthroplasty: a systematic review of the literature in the English language until the end of 2003. Journal of Bone and Joint Surgery. 2005; 87(4): 437-444.
Mokbel A, El-Tookhy O, Shamaa AA, et al. Homing and efficacy of intra-articular injection of autologous mesenchymal stem cells in experimental chondral defects in dogs. Clin Exp Rheumatol 2011; 29: 275-84.
Murphy J M, Fink D J, Hunziker EB, et al. Stem cell therapy in a caprine model of osteoarthritis. Arthritis Rheum 2003; 48: 3464-74.
Nguyen D, Proper S I W, MacDermid J C, King G J W, Faber K J. Functional outcomes of arthroscopic capsular release of the elbow. Arthroscopy. 2006; 22(8): 842-849.
Pettine K A, Murphy M B, Suzuki R K, Sand T T (2015) Percutaneous injection of Autologous bone marrow concentrate significantly reduces lumbar discogenic pain through twelve months, Stem Cells 33: 146-56.
Pettine K P, Dordevic M. The Biologic Treatment of Osteoarthritis with Mesenchymal Stem Cell Exosomes: The Future is now. J Stem Cell Res Dev Ther. 2019; 1-5.
Pettine K P, Dordevic M. Tibial Metaphyseal Injection with Bone Marrow Concentrate to Treat Knee Arthritis. American J Stem Cell Res Ther. 2018; 2(1): 5-10.
Pettine K P, Suzuki R. Autogenous Bone Marrow Concentrate for the treatment of osteoarthritis of the knee, hip and shoulder in former NFL players. J Stem Cell Res Ther. 2018; 4(1): 9-13.
Philippon M J. Nepple J J, Campbell K J, Dornan G J, Jansson K S, LaPrade R F, et al. The hip fluid seal-part 1: the effect of an acetabularlabral tear, repair, resection, and reconstruction on hip fluid pressurization. Knee Surg Sports Traumatol Arthrosc. 2014; 22(4): 722-9.
Savoie F H, Nunley P D, Field L. D. Arthroscopic management of the arthritic elbow: indications, technique, and results. Journal of Shoulder and Elbow Surgery. 1999; 8(3): 214-219.
Seldes R M, Tan V, Hunt J, Katz M, Winiarsky R, Fitzgerald RH Jr. Anatomy, histologic features, and vascularity of the adult acetabular labrum. Clin Orthop Relat Res. 2001; 382: 232-40.
Smith R K W, Korda M, Blunn G W, Goodship A E (2003) Isolation and implantation of autologous equine mesenchymal stem cells from bone marrow into the superficial digital flexor tendon as a potential novel treatment. Equine Veterinary Journal. 35: 99-102.
Smith R K W. Mesenchymal Stem Cell Therapy in Equine Tendinopathy. Disabil Rehabil (2008) 30: 20-22, 1752-1758.
Vangsness C T, Farr J, Boyd J, Dellaero. Adult human mesenchymal stem cells delivered via intra-articular injection to the knee following partial medial meniscectomy: a randomized, double-blind controlled study. JBJS. 2014: 90-98.
Zhang S, Chuah S J, Lai R C, Hoi J H P, Lim S K, Toh W S. MSC Exosomes Mediate Cartilage Repair by Enhancing Proliferation, Attenuating Apoptosis and Modulating Immune Reactivity. Biomaterials. 2018 February; 156: 16-27.
Zhao L, Kaye A D. Stem Cells for the Treatment of Knee Osteoarthritis: A Comprehensive Review. Pain Physician. 2018; 21: 229-241.

1.-6. (canceled)

7. A method of preventing a disease, disorder, injury or symptoms thereof affecting one or more joints in a subject comprising administering to a subject a therapeutically effective amount of a mesenchymal stem cell (MSC) exosome preparation.

8. The method of claim 7, wherein disease or disorder comprises juvenile arthritis, psoriatic arthritis, infectious arthritis, rheumatoid arthritis, ankylosing spondylitis, gout, bursitis, tendinosis, and/or tendonitis.

9. The method of claim 7, wherein the joint comprises the ankle, knee, hip, wrist, elbow, shoulder, knuckle, and/or neck.

10. The method of claim 7, wherein the MSC exosome preparation is administered to each joint affected by the disorder, disease, or injury.

11. The method of claim 7, wherein the MSC exosome preparation further comprises growth factors obtained from MSC.

12. The method of claim 7, wherein the MSC exosomes are administered via injection, MSC exosome carrying scaffold, hydrogel, and/or topical cream or salve.

13. A method of treating, inhibiting, reducing, and/or ameliorating a disease, disorder, injury or symptoms thereof affecting one or more joints in a subject comprising administering to a subject a therapeutically effective amount of a mesenchymal stem cell (MSC) exosome preparation, wherein the symptoms comprise a tear in a piece of tissue.

14. The method of claim 13, wherein disease or disorder comprises sprain, labral tear, tear of a tendon, and/or tear of a ligament.

15. The method of claim 13, wherein the joint comprises the ankle, knee, hip, wrist, elbow, shoulder, knuckle, and/or neck.

16. The method of claim 13, wherein the MSC exosome preparation is administered to each joint affected by the disorder, disease, or injury.

17. The method of claim 13, wherein the MSC exosome preparation further comprises growth factors obtained from MSC.

18. The method of claim 13, wherein the MSC exosomes are administered via injection, MSC exosome carrying scaffold, hydrogel, and/or topical cream or salve.

19. A method of preventing a disease, disorder, injury or symptoms thereof affecting one or more joints in a subject comprising administering to a subject a therapeutically effective amount of a mesenchymal stem cell (MSC) exosome preparation, wherein the symptoms comprise a tear in a piece of tissue.

20. The method of claim 19, wherein disease or disorder comprises sprain, labral tear, tear of a tendon, and/or tear of a ligament.

21. The method of claim 19, wherein the joint comprises the ankle, knee, hip, wrist, elbow, shoulder, knuckle, and/or neck.

22. The method of claim 19, wherein the MSC exosome preparation is administered to each joint affected by the disorder, disease, or injury.

23. The method of claim 19, wherein the MSC exosome preparation further comprises growth factors obtained from MSC.

24. The method of claim 19, wherein the MSC exosomes are administered via injection, MSC exosome carrying scaffold, hydrogel, and/or topical cream or salve.
